(19) United States
(12) Patent Application Publication
(10) Pub. No.: US 2023/0309860 A1
(43) Pub. Date: Oct. 5, 2023

(54) METHOD OF DETECTING AND TRACKING BLINK AND BLINK PATTERNS USING BIOPOTENTIAL SENSORS

(71) Applicant: AAVAA Inc., Montreal (CA)

(72) Inventor: Naeem Komeilipoor, Montreal (CA)

(21) Appl. No.: 18/022,665

(22) PCT Filed: Jul. 26, 2022
(86) PCT No.: PCT/IB2022/056905

§ 371 Date: Feb. 22, 2023

(30) Foreign Application Priority Data
Aug. 3, 2021 (CA) 3126636

(51) Int. Cl. A61B 5/11; A61B 5/0205; A61B 5/00; A61F 4/00

(52) U.S. Cl. CPC A61B 5/1103; A61B 5/1126; A61B 5/0205; A61B 5/7278; A61B 5/7267; A61F 4/00; A61B 5/291

(57) ABSTRACT

A method for detecting and tracking blink and blink patterns of a user from a headworn device, the method including placing an electronic device with a housing on a head of the user, placing one or more biopotential sensors of the housing in contact with skin of the user, detecting, using the biopotential sensors, signals indicative of blink or blink patterns of the user, processing the signals by a processing unit configured to identify blink of the user, and inputting the blink signals into a model capable of decoding gaze and eyelid motion in real-time to understand the attention, intention, and states of the user.

### FIELD OF THE DESCRIPTION

The following relates to a method of detecting and tracking blink and blink patterns without occluding the field of vision of a user from non-invasive biopotential signals on the head of the user.

### BACKGROUND

Eye motion and blinks have proven to be a reliable source of communication for understanding the intention and attentional focus of a user. Even for people with extreme motor disabilities, such as complete paralysis of nearly all voluntary muscles in the body, the ability to control eye movements and blinks are still preserved (Hori, et al, 2004). More generally, by understanding the intention and attentional focus of a user, eye motion and blinks can be even used for various applications in user interfacing with electronic devices, such as for aiding in the communication even for people without motor disabilities.

Blinking is a major aspect of eye motion. Eye blinks, defined by the shutting and opening motion of the eyelids, is facilitated by two muscle groups: to shut the eyelids, the circumferential orbicularis oculi muscle is used; and to open the eyelids, the levator palpebrae superioris and the Muller's muscle groups are used (Tong, et al., 2020, Abdelhady, et al., 2020). Eyelid movements, referred to as eyelid contractions, twitches, or blinks, can occur spontaneously, reflexively, or voluntarily. Spontaneous blinking occurs without either external stimuli or internal effort, and is conducted in the premotor brain stem area of the brain. Typically, spontaneous blinks occur at a rate of ten blinks per minute, though in the presence of visual concentration, this can reduce to three or four blinks per minute (Portelo, et al, 2013). Second, reflex blinking is the eyelid motion in conjunction with external stimuli. Reflex blinking occurs when the eyelids are responding to protect the anterior surface of the eye from an external event, which can be either tactile, optical, or auditory stimuli. This kind of blink generally also occurs unintentionally. Finally, the intentional effort of shutting and opening the eyes is called voluntary blinking. Voluntary blink controls originate in the cerebrum. Consequently, voluntary blinking has larger amplitudes and occurs slower than reflex or spontaneous blinks (Bologna, et al., 2009). Because of the unique properties of each kind of blink, they can be differentiated from one another, and can provide many insights to the state of the user, which can in turn be used as an interface between a user and device.

This invention uses the electrical potential generated by the eyelids and its related muscles to detect blinks, classify the type of the blink, determine characteristics of blink, and track patterns of blink in real-time, to be used as a Brain-Computer Interface for electronic devices to control a wide variety of applications, including consumer applications, medical, therapy, and rehabilitation applications, and others.

In conjunction with identifying blinks, another useful input to understanding the attention and intention of a user is eye motion. In fact, in the past decade, strategies have evolved to detect eye motion, such as one that is also conducive for detecting blinks, electrooculography (EOG) (Sato, et al., 2010). This technique detects eye-gaze direction by measuring eye potential (electrooculogram) generated by a positive charge in the cornea and a negative charge in the retina. EOG has several advantages over traditional video-based eye tracking methods, such as being independent from outside light, the shape of the eye, or the opening state of the eye (Chang, 2019; Aimone, et al., 2019).

To this end, eye gaze and blink detection are complementary modules to a full system design incorporating brain, eye, head, and body information to determine the attention and intention of a user (Komeilipoor, 2021). For instance, blinks have a significant effect in the presence of electroencephalography (EEG). EEG reflects changes in the electric potential distribution from across the scalp of a user, providing insights to the complex electrical activity of the brain. Due to the sensitivity of these signals, minute electrophysiological changes, such as from eye motion or blink muscles of a user, result in distinct noise artefacts (Virtanen, et al., 2006; Chang, et al., 2016). Due to the relatively significant amplitude of eye and eyelid movements, these are often identified and removed from EEG signals for neuroscience applications. One such method is independent component analysis (ICA), which decomposes signals into independent components (ICs) to isolate cerebral and artifactual sources, and then reconstructs the clean signal by discarding ICs containing artifacts (Li, et al., 2006). Similarly, rather than using methods such as ICA to remove blink artefacts, these methods can be used to identify and isolate eye and blink signals in the EEG signals, which can then be repurposed to provide information regarding the blink patterns of a user.

Although eye information, such as gaze direction and blinking, can inform a device about the attention direction and state of the wearer, knowledge of the head and trunk orientation of a user can provide a device with additional complementary information about the action and intention of the user in the environment, and thus provide a more comprehensive understanding of the user as they interact with their device and environment (Komeilipoor, 2021).

By understanding these signals, deliberate voluntary blinks or sequences of voluntary blinks can be used to replace more traditional user interface methods. The wearable electronic device may process EOG measurements together with eye blink detection to enable new dimensions of control, such as recognizing where the user's eye direction is, and then interpreting an intentional blink as a selection command for navigating a menu. To conceptualize this, imagine a display interface offering several options to the user. Eye motion can be used to detect which item is of interest to the user on the display, and then an intentional blink could be used in the place of a mouse click to select the item. In another example, a double-blink or blink with a certain duration, or time to complete a blink cycle, intensity, or the physical exertion used to perform the blinks, or velocity, or speed at which the blink is performed, can be assigned to execute specified tasks, such as substitutes for mouse clicks or voice commands. In addition, sequences of unilateral blinks, such as blinking with the left eye or blinking with the right eye, can produce another dimension of commands. For one example, a left link can be used to repeat a song, while a right link can be used to skip a track. Furthermore, additional information can be understood from a blink signal, since blink speed can be affected by elements such as fatigue, eye injury, medication, and disease. By monitoring these and similar characteristics, it can be determined whether the user is fatigued, ill, distracted, etc. A skilled reader will understand that various other implementations are possible.

### SUMMARY OF THE DESCRIPTION

In one aspect, herein is provided a method for detecting and tracking blink and blink patterns comprising a body configured to engage a user's head, at least one sensor mounted to the body for collecting blink, electroencephalogram and/or electrooculogram and/or electromyogram signals; at least one sensor for collecting additional signals from the user or the environment; and a processor configured to use the electroencephalogram and/or electrooculogram and/or electromyogram and/or blink signals and with or without environment signals as input to linear or non-linear analog and digital neural network algorithms to determine auditory or visual attention or intention of the user in real time (Komeilipoor, 2021).

In a further aspect, the at least one sensor comprises one or more in-ear sensors and/or one or more around-ear sensors.

In a further aspect, the device connected wirelessly to a network of devices such as a cell phone, and/or smart watch, and/or smart glasses, and/or other electronic device for collecting signals from the environment.

In a further aspect, the electroencephalogram and/or electrooculogram and/or electromyogram signals are used as inputs into linear and/or non-linear models to detect the eye gaze and eyelid motion of the user.

In a further aspect, the headworn device comprises a head orientation sensor for determining the relative orientation between the eye gaze, head, and trunk.

In a further aspect, the head orientation sensor is a combination of one or more of an accelerometer, and/or gyroscope, and/or magnetometer.

In a further aspect, additional biosignals can be collected from the user, including but not limited to temperature, blood pressure, heart rate, oxygenation level, skin conductance, etc.

In a further aspect, the blink signals can be integrated with one or a combination of the EEG, EOG, EMG, accelerometer, gyroscope, magnetometer, bio-signals, and environmental signals to provide a holistic understanding of the user and the environment.

In a further aspect, the device system comprises the headworn device connected through wireless transmission with other sensors from an external electronic device to provide additional information concerning the state and/or the environment of the user, such as cameras, vibration sensors, equipment sensors, microphones, etc.

In a further aspect, for the recording of biopotential signals from the head of the user, since placement of electrodes around or near the eye of a user is not suitable for consumer applications other than on eye-worn devices such as goggles, glasses, etc., biopotential sensors may be placed on earworn devices that are more discreet and still capable of providing relevant blink information. Thus, a hearing device is described, comprising: a body configured to engage a user's ear; at least one ear sensor mounted to said body for obtaining at least one biosignal indicative of the user's attention and eye and eyelid motion; and a processing unit adapted to use the at least one biosignal to determine the attention or intention of the user in real-time (Klappert, et al., 2013).

In a further aspect, the at least one biosignal is chosen from the group consisting of electroencephalogram (EEG), electrooculogram (EOG), electromyogram (EMG), blink, accelerometer, gyroscope, and/or magnetometer signals.

In a further aspect, the at least one biosignal is used to determine, in real time, at least one of auditory attention of the user, visual attentional direction of the user, information of the user's blinking, and physical orientations of the user's head, gaze, and trunk, and intention of the user.

In a further aspect, the at least one ear sensor comprises at least one in-ear sensor and/or around-ear sensor used to obtain the at least one biosignal chosen from the group consisting of EEG, EOG and EMG, from which blink can be derived.

In a further aspect, obtaining one or more of the EEG, EOG and/or EMG signals of the user comprises: obtaining a change in electrical potential of the user via a non-invasive recording from at least one ear sensor comprising a combination of one or more in-ear sensors and/or around-ear sensors.

In a further aspect, the at least one biosignal is an EEG signal from which a blink signal can be derived, and the signal is used as input into linear and/or non-linear models to determine the auditory and visual attention of the user and/or understand the intention of the user based on bio-signals and blink signals.

In a further aspect, the at least one biosignal is collected from sensors in or around both the right ear and left ear of a user and the signals from the right ear and/or signals from the left ear are used as input into linear and/or non-linear models to identify eye gaze and blink.

In a further aspect, the device can be configured to provide an EOG and/or blink signal from in and/or around the ear representative of eye gaze and blink is determined based on the left and right amplified voltages Vleft and Vright.

In a further aspect, the device can be configured to provide that the EOG and/or blink signal is a function (f) of one or more or an average of multiple signals from the left amplified voltages Vleft, signal=f(Vleft), or right amplified voltages Vright, signal=f(Vright) or from the difference between one or more or an average of multiple signals of the left and right amplified voltages Vleft and Vright, signal=f(Vleft−Vright).

In a further aspect, the device can comprise a processing unit configured to provide an EOG control signal and/or blink control signal for controlling a function of said at least one headworn device based on said EOG signals.

In a further aspect, linear and/or non-linear models are applied to identify the horizontal and vertical movement of the user's eye and provide gaze angles in a fixed coordinate system.

In a further aspect, the linear and/or non-linear models use linear and non-linear activation functions respectively embedded in a shallow or deep neural network algorithm.

In a further aspect, at least one of the hearing devices comprises a beamformer unit, and wherein said at least one hearing device is configured to steer and select the beamformer angle of maximum sensitivity towards the gaze direction.

In a further aspect, the device can provide absolute coordinates of a sound source to identify individual attended sound signals from the plurality of sound signals.

In a further aspect, the device can further comprise a processing unit configured to provide a control signal to direct sound processing based on a computed head rotation angle of the user in a fixed coordinate system using said linear and/or non-linear models to be used in conjunction with blink commands.

In a further aspect, the device can further comprise EMG in-ear and/or around-ear sensors for determining neck orientation angles of the user relative to the trunk in a fixed coordinate system to be used in conjunction with blink commands.

In a further aspect, the at least one measured biosignal is chosen from the group consisting of one or more of electroencephalogram (EEG), electrooculogram (EOG), electromyogram (EMG), blink, accelerometer, gyroscope, magnetometer, galvanic skin conductance, temperature, heart rate, oxygenation level, electrocardiography (ECG), etc.

In a further aspect, the at least one biosignal is measured using sensors in and/or around the ear.

In a further aspect, the at least one biosignal is used to determine, in real time, at least one of auditory attention of the user, visual attentional direction of the user, blink signals of the user, and physical orientations of the user's head, gaze, and trunk.

In a further aspect, the method may comprise: processing the selected at least one of the plurality of separated sound signals based on the selected sound source derived from the said auditory attention identification method and confirming by blink, including performing one or more of: amplifying the selected one or more of the plurality of separated signals, or suppressing at least one of the non-selected sound signals from the sound signals.

In a further aspect, the neural-network-based sound-separation processing is applied to the mixed sound signal from the multiple sound sources in isolation or in combination with at least one EEG signal recorded from either the left and/or right ear.

In a further aspect, the at least one biosignal is an average of multiple of the EOG signals collected from the left and right sides of the body which results in amplified voltages Vleft and Vright are used after being band pass filtered and/or feature extracted with said measuring device or system, establishing a measurement of the signal power in different frequency bands, mean, standard deviation, slope, average velocity, maximum velocity, average acceleration, peak-to-peak amplitude, maximum amplitude, and/or time-to-peak, from which blink signals can be derived.

In a further aspect, the device can receive through transmission additional signals from external electronic devices to understand the conditions of the state and attention of the user and the environment.

In a further aspect, the signals can be integrated with signals from an electronic device or system to provide a comprehensive understanding of the user and their environment; said integration being performed using a sensor fusion method for integrating a combination of said auditory attention data, gaze direction data, blink data, gaze-head-trunk orientation data, location data, sound data, separated sounds, raw EEG, EOG, and/or EMG signals, blink signals, and inertial data, and/or signals from external electronic devices that provide additional information concerning the environment of the user, such as visual data, etc. to identify and provide the focus of attention of the user and perform other attention-related tasks.

In a further aspect, the sensor fusion method can be used to furthermore improve the data, e.g., to reduce drift, increase robustness, and denoise speech signals, EOG signals, blink signals, or other signals.

In a further aspect, the device includes other biopotential sensing modalities, including one or more of functional near infrared spectroscopy (fNIRS), magnetoencephalography (MEG), optical pumped magnetoencephalography (OP-MEG), giant magnetoimpedance (GMI), and functional ultrasound (fUS), wherein the processing unit is adapted to process one or more of fNIRS, MEG, OP-MEG, GMI, fUS, EEG, EOG, EMG, accelerometer, gyroscope, and magnetometer signals and auditory signals to: determine in real time auditory attention of the user; determine the visual attentional direction of the user; determine blink motions of the user, determine physical orientations of the user's head, gaze, and trunk; the device to obtain one or more of fNIRS, MEG, OP-MEG, GMI, fUS, EEG, EOG, EMG, electrocardiogram (ECG), temperature, accelerometer, gyroscope, and magnetometer signals of the user indicative of the attention and/or intention of the user.

In a further aspect, the blink with or without the one or more bio-signals can provide information regarding conditions of the user's physical health, including fatigue, injury, illness, disease, etc. (Lisy, et al., 2014)

In a further aspect, the blink data can provide additional information about the state of the opening of the eye.

### BRIEF DESCRIPTION OF THE FIGURES

Embodiments will now be described by way of example only with reference to the appended drawings wherein:

FIG. 1 is a block diagram of the input 101, output 103 and processing unit 102 of the electronic device, according to one embodiment of the present subject matter.

FIG. 2 depicts a schematic illustration of an embodiment of an electronic device 201 and its integration with a smartphone 203 and a smartwatch 202 where blink can be used to command different functions on said connected devices.

FIG. 3 depicts a schematic illustration of a single electronic device to be placed around and in the ears of a user 300 including in-ear electrodes 301, around-ear electrodes 302, and both omnidirectional 304 and directional 303 microphones. The in-ear and around-ear electrodes collect EEG, EOG, EMG, and blink data from the user.

### DETAILED DESCRIPTION

The terms “comprise”, “comprises”, “comprised” or “comprising” may be used in the present description. As used herein (including the specification and/or the claims), these terms are to be interpreted as specifying the presence of the stated features, integers, steps, or components, but not as precluding the presence of one or more other feature, integer, step, component, or a group thereof as would be apparent to persons having ordinary skill in the relevant art. Thus, the term “comprising” as used in this specification means “consisting at least in part of. When interpreting statements in this specification that include that term, the features, prefaced by that term in each statement, all need to be present but other features can also be present. Related terms such as “comprise” and “comprised” are to be interpreted in the same manner.

Unless stated otherwise herein, the article “a” when used to identify any element is not intended to constitute a limitation of just one and will, instead, be understood to mean “at least one” or “one or more”.

The following relates to a method of detecting and tracking blink and blink patterns that use signal processing methods and machine learning approaches such as analog and digital artificial neural networks to achieve a combination of detecting blink and blink patterns of a user and using it in combination with user attention detection, which are all done by employing the user's brain signals and other biosignals in conjunction with information gathered from the environment, including auditory data and data from other sensors providing relevant information on the environment of the user.

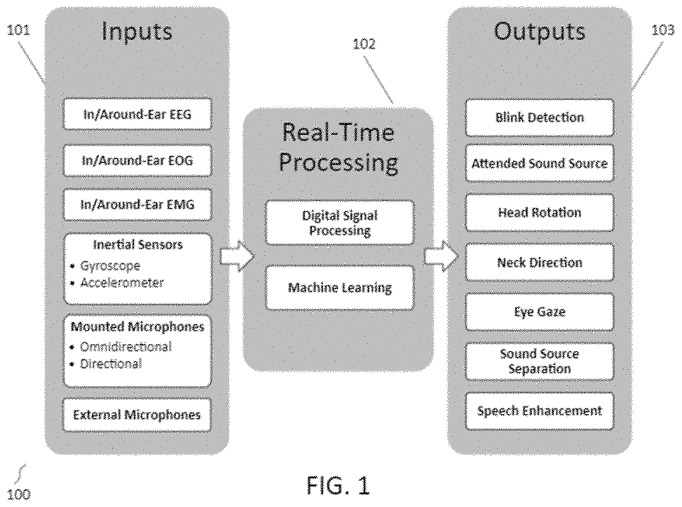

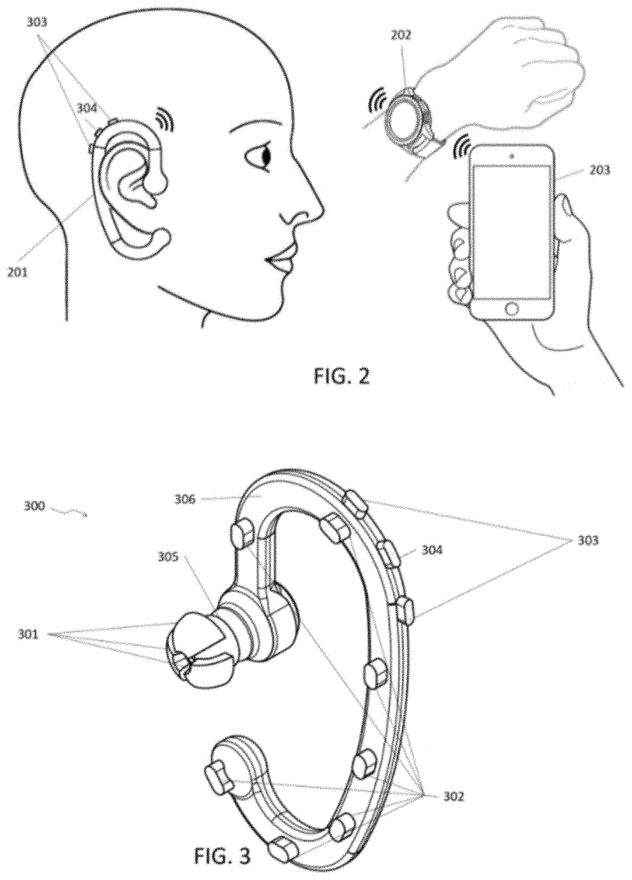

FIG. 1 is a block diagram of a system 100, the outputs 103 of which can be used for a variety of different applications. In one embodiment, a hearing device 300, shown in FIG. 3 is provided as a mounting device for all the sensors or inputs 101. The signals from these sensors are used as input into a processor for real-time processing 102 including, at least signal processing and machine learning algorithms. Depending on which inputs 101 and algorithms are used, a variety of outputs 103 are possible. These outputs 103 could include, but are not limited to, blink detection, attention, attention direction, intention, and preset electronic commands.

An embodiment wherein a multimodal blink detection headworn device 201 is configured to collect the input signals will be discussed below.

### Earworn Device

Personal listening devices and earworn devices are limited in their ability to understand the attention or intention of a user. This limitation prevents them from responding dynamically to a user's preference, and consequently, provides only a stagnant listening experience, requiring the user to physically interfere with their device when different settings are needed or commands are required to understand the intention of a user, such as pausing a song, turning up the volume, or other commands. The image 300, shown in FIG. 3 is a representation of a device that incorporates a new form of user interface for a device to interact with a user: eye motion and blink detection. The eye motion provides information regarding the attention of the user, and the eyelid motion provides information relevant to the intention of the user. These are determined from a plurality of sensors. In a preferred embodiment of the device 300, a plurality of different measurement devices are incorporated into the device, including one or a plurality of in-ear sensors 301, one or a plurality of around-ear 302 versatile dry electrodes, and one or more microphones or microphone arrays preferably consisting of directional 303 and omnidirectional 304 microphones. Furthermore, accelerometer, and/or gyroscope, and/or magnetometer sensors and/or other bio-signal sensors may also be included.

The in-ear sensors and around-ear sensors are preferably made of conductive material, including, but not limited to, metals or polymers with the ability to measure bioelectric signals of the user with whom they have contact. These sensors could be capable of measuring at least one of a variety of signals, including signals such as electroencephalogram (EEG), electromyogram (EMG), electrooculogram (EOG), and blink signals. In the example embodiment shown, 3 in-ear sensors 301 are located at the end of an extension support 305 that extends inwardly from the body 306 of the hearing device 300. When in use, the in-ear sensors 301 are preferably electrodes, and engage the ear canal of the user's ear. In a preferred embodiment, there could be one or multiple in-ear sensors as could be appreciated by a person skilled in the art. Said in-ear and around-ear sensors may also be in the form of other brain imaging modalities, such as used for functional near infrared spectroscopy (fNIRS), magnetoencephalography (MEG), optical pumped magnetoencephalography (OP-MEG), giant magnetoimpedance (GMI), and functional ultrasound (fUS), which can detect the brain's response to different stimuli, as well as the inclusion of blink signals in the brain signal, as can be appreciated by a person skilled in the art.

The body 306 of the device 300, further includes a plurality of around ear sensors 302. These sensors are preferably mounted on a back surface of the body 306 in such a fashion that they contact the user's head. In a preferred embodiment shown, there are 7 around ear sensors, a person skilled in the art would understand that the number of around ear sensors could vary.

Real-time Visual Attention Decoding and Eye Blink Detection via Eye Gaze Tracking Using In- and Around-Ear Electrodes

Current eye gaze and blink tracking methods based on an electrooculogram either use signals recorded from around the eyes or a limited number of signals inside the ear. The system described herein detects a gaze direction and blink of a user based on electrooculogram and electromyogram signals recorded from the in-ear sensors 301 and around-ear sensors 302. The additional signals from the around-ear sensors 302 lead to an increase in signal quality and thus enhances the accuracy and resolution of eye gaze and blink detection. The horizontal and vertical gaze direction (right, left, up, down and center) as well as the angle of the gaze relative to the head is computed based on approximations of voltage ratios and/or subtraction or other interactions between and within the right and left in-ear sensors 301 and around-ear sensors 302. By using sensors located on both the left ear and right ear of the user, the signal quality can be increased by subtracting the signals from one another in order to identify distortions that appear as common artefacts between the signals which represent additional signal noise. The extraction of horizontal and vertical direction and gaze angles is decoded using thresholding methods as well as linear and non-linear models, including but not limited to, Linear and Logistic Regressions, Naive Bayes, Support Vector Machine, K-nearest Neighbors, Decision Tree and Random Forest and Neural Network models such as convolutional neural networks, and from these signals, additional information such as electromyography, can be gathered, which is used to determine head rotation, trunk orientation, and blink, providing an understanding of the absolute gaze direction in the user's field as well as unilateral and bilateral blink patterns, and thus the sensors behind the ear provide additional information about the state of the user.

### Attention Tracking and Intention Detection Based on Sensor Fusion Models

Sensor fusion models are algorithms that combine series of noisy data in order to produce estimates of unknown variables. By estimating a probability distribution based on these combined data, these estimates tend to be more accurate than those based on a single measurement alone. One example of these is the Kalman filter suited for temporal applications, where the probability distributions based on the data is segmented by time for real-life applications. By implementing the sensor fusion model into the headworn device, in addition to integrating the data from all the sensors of the device, the system can be modified to include the information from sensors external to those provided on the hearing device itself, including information from sensors that provide additional knowledge of the environment of the user, such as visual, or other sensory information. These can be combined with the information of the user and the user's attention and intention provided from the headworn device in a signal fusion method for filtering a combination of one or more of said blink data, auditory attention data, gaze direction data, gaze-head-trunk orientation data, location data, sound data, separated sounds, raw EEG, EOG, EMG signal(s), and/or said combined location data, in conjunction with one or more of these external signals from external electronic devices that provide additional information concerning the environment of the user. This fusion of multiple on-device and off-device sensors can be used to provide a holistic understanding of the environment and state of the user, identify the user's attention and intention, and perform appropriate functions. Furthermore, these data may be further improved by the use of sensor fusion methods, e.g., to reduce drift (e.g. Manabe & Fukamoto; 2010), increase robustness, and denoise speech signals or other signals.

### Alternative Embodiments

The above-described principles can be modified according to the following: the device can be a network of a device with one or more individual electrodes placed on the head or face of the user; the device can be miniaturized into a smaller package such as an in-ear hearing device; or the device can be enlarged to be suitable for a headphone, glasses frame, virtual or augmented reality headset, or helmet unit.

The network of a device with one or more individual electrodes includes: a device with a housing unit and processor capable, and one or more nodes that may or may not be spatially separate from the device, and that contain electrodes for the collection of EEG, EOG, EMG, and blink data from the head or face of the user. Additional nodes may be added to the device to increase the signal-to-noise ratio of the collected signals.

The smaller package resides in an embodiment of an in-ear hearing device that includes: one or more in-ear dry electrodes for the collection of EEG, EOG, EMG, and blink data from the ear canal of the user, microphones placed on an outward face of the body of the hearable device, as well as accelerometer, gyroscope, magnetometer, and other bio-signal sensors embedded in the device (Pontoppidan, et al., 2020). Additional miniaturized in-ear dry electrode layers can be added into the device along additional planes of skin contact in the ear to increase the signal-to-noise ratio of the collected signals while maintaining the same effective areas as the inserted earphones.

The larger package resides in an embodiment of a stand-alone headphone unit, glasses frame, virtual or augmented reality headset, or a headphone unit that is incorporated into a helmet including the following elements: around-ear electrodes to be placed in or around the ear of the user that collect EEG, EOG, EMG, and blink data, multiple dry electrodes on the inside of the unit against the skin of the user to collect signals from the scalp, microphones placed both on the outer surface of the unit and/or mounted on the body of a consumer electronic device such as smartphones, smart glasses, virtual or augmented reality headsets, smart watches, or other consumer devices, as well as accelerometer, gyroscope, magnetometer, and other bio-signal sensors embedded in the device.

The principles, devices, and systems described herein have applications in many areas that involve detecting the visual and auditory attention of the user, direction of gaze, head, and trunk orientation of the user, intention and blink patterns of the user, as well as additional features. An advantage this device brings over alternatives is that it can detect the behavior and attention and intention of the user and perform related functions all in a single package by employing several EEG, EOG, EMG, blink dry electrodes, accelerometer, gyroscope, and magnetometer sensors, microphones and additional external sensors in wirelessly-connected devices including but not limited to smartphones, tablets, smart glasses frames, virtual or augmented reality headsets, smartwatches, and helmets.

Additional applications include but are not limited to Communication for People with Mobility Disorders, Signaling an Audio Device, Human-Computer Interaction for Electronic Devices, Automotive and Heavy Machinery, and Augmented Reality (AR) or Virtual Reality (VR), each of which is discussed below.

Communication Medium for People with Mobility Disorders

Using the principles described above, information of the user's eye gaze direction and blink patterns can be interpreted to provide means to operate a computer or electronic device, such as an electric wheelchair, to people who have limited mobility, such as full body paralysis. Direction of motion on a screen or in a room can be interpreted from the direction of the user's gaze, and intention, such as selecting, operating, removing, etc. can be determined through the blink or blink pattern of the user.

### Non-verbal, Non-Tactile Method for Signaling an Audio Device

Using the principles described above, the detected blink and blink patterns, as well as attentional signals from the user, can be used to provide commands to personal audio devices, such as but not limited to mobile phones, earphones, headphones, hearing aids, cochlear implants, regarding the intention of the user. In order to facilitate a better speech enhancement paradigm, speech enhancement audio processing can be directed by gaze direction, and a user can confirm or correct the selection of enhanced sounds using blinks. Furthermore, the blink interface can be used to replace other shortcut commands, such as controlling volume, changing preset settings, etc.

### Human-Computer Interaction to Facilitate Commands to Electronic Devices and Wirelessly Connected Devices

Using the principles described above, the detected blink and blink patterns, as well as attentional signals from the user, can be used to provide insights and commands to electronic devices, such as but not limited to mobile phones, smart watches, electric wheelchairs, audio devices, earphones, headphones, hearing aids, cochlear implants, computers, appliances, gaming devices, augmented reality devices, virtual reality devices, extended reality devices, machinery, vehicles, electronics connected by wireless connectivity, regarding the intention of the user. The blink signals can be assigned to predesignated commands as an alternative to conventional user interaction modalities, such as mouse clicks, keyboards, touchscreens, touchpads, etc. by using the gaze of the user to translate to a new location on a display, and then using preset commands associated with blinks to perform different operations, including but not limited to selecting different desired sounds, confirming an action, dismissing a notification, changing audio settings, answering a phone, choosing between connected devices, switching between pre-programmed settings, etc.

### Automotive and Heavy Machinery

Using the principles described above, information on the state of a driver can be interpreted, including, but not limited to, driver's or operator's attention, distraction, intentional, fatigue, and mental and physical safety level.

Using the attention tracking and intention detecting models, hands-free control can be provided to the driver or operator to reduce distraction.

Using blink analysis and gaze tracking, a driver's or operator's eye gaze can be tracked both during the day and night independently of lighting conditions or information provided by any eye-tracking camera.

Additional information on the state of the vehicle or environment collected by the sensors of the vehicle or system can be fused with the information on the state of the driver or operator to provide a more holistic understanding of the driving conditions or environment for further safety or attention applications.

Using the EEG, EOG, EMG, blink signals recorded from in-ear electrodes 301 and around-ear electrodes, 302 shown in FIG. 3, the fatigue level of the driver can be predicted from monitoring both the eye conditions and mental conditions of a driver or operator.

All the points described above contribute to the understanding of the level of driver's or operator's attention to the road conditions.

### Augmented Reality (AR) or Virtual Reality (VR)

Using the principles described above, information about the user of VR/AR is interpreted, including, but not limited to, the user's attention to visual and auditory stimuli in their virtual environment and the user's blink as a command to interact with the virtual environment.

Although the above description includes reference to certain specific embodiments, various modifications thereof will be apparent to those skilled in the art. Any examples provided herein are included solely for the purpose of illustration and are not intended to be limiting in any way. Any drawings provided herein are solely for the purpose of illustrating various aspects of the description and are not intended to be drawn to scale or to be limiting in any way. The scope of the claims appended hereto should not be limited by the preferred embodiments set forth in the above description but should be given the broadest interpretation consistent with the present specification as a whole. The disclosures of all prior art recited herein are incorporated herein by reference in their entirety.

### REFERENCES

Hori J., Sakano K., et al. Development of communication supporting device controlled by eye movements and voluntary eye blink. Conf IEEE Eng Med Biol Soc. 2004; 6:4302-5.

Tong J, Lopez M J, Patel B C. Anatomy, Head and Neck, Eye Orbicularis Oculi Muscle. [Updated 2020 Jul. 27]. In: StatPearls [Internet]. Treasure Island (FL): StatPearls Publishing; 2021 January. Available from: https://www.ncbi.nlm.nih.gov/books/NBK441907/

Abdelhady A, Patel B C. Anatomy, Head and Neck, Eye Superior Tarsal Muscle (Mullers Muscle) [Updated 2020 Sep. 16]. In: StatPearls [Internet]. Treasure Island (FL): StatPearls Publishing; 2021 January. Available from: https://www.ncbi.nlm.nih.gov/books/NBK540964/

Portello, J., Rosenfield, M., Chu, C. (2013). Blink Rate, Incomplete Blinks and Computer Vision Syndrome. Optometry and vision science: official publication of the American Academy of Optometry. 90. 10.1097/OPX.0b013e31828f09a7.

Bologna, M., Agostino, R., Gregori, B., Belvisi, D., Ottaviani, D., Colosimo, C., Fabbrini, G., Berardelli, A., Voluntary, spontaneous and reflex blinking in patients with clinically probable progressive supranuclear palsy, Brain, Volume 132, Issue 2, February 2009, Pages 502-510, https://doi.org/10.1093/brain/awn317

Chang, W. D. Electrooculograms for human-computer interaction: A review. Sensors (Basel, Switzerland). 2019. https://pubmed.ncbi.nlm.nih.gov/31207949/.

Virtanen, J., Rantala, B., Virtanen, S. I. J. (2006). Detection of artifacts in bioelectric signals.

Chang, W. D., Cha, H. S., Kim, K., & Im, C. H. (2016). Detection of eye blink artifacts from single prefrontal channel electroencephalogram. Computer methods and programs in biomedicine, 124, 19-30. https://doi.orci/10.1016/j.cmpb.2015.10.011

Li, Y., Ma, Z., Lu, W., & Li, Y. (2006). Automatic removal of the eye blink artifact from EEG using an ICA-based template matching approach. Physiological measurement, 27(4), 425-436. https://doi.org/10.1088/0967-3334/27/4/008

Komeilipoor, N. (2021). MULTIMODAL HEARING ASSISTANCE DEVICES AND SYSTEMS. International Application No. CA2021050730. Gatineau, QC: Canadian Intellectual Property Office.

Smit, A. E. (2009). Blinking and the brain: Pathways and pathology (dissertation). Haveka, Rotterdam, The Netherlands.

Lisy, F. J., Opperman, A., Dashevsky, D. D. (2014). Head-mounted physiological signal monitoring system, devices and methods.

Pontoppidan, N. H., Lunner, T., Pedersen, M. S., Hauschultz, L. I., Koch, P., Naylor, G., Petersen, E. B. (2020). Hearing assistance device with brain computer interface.

Aimone, C. A., Garten, A. S., Coleman, T., Pino, L. G., Vidyarthi, K. J. M., Baranowski, P. H., Chabior, M. A., Chong, T., Rupsingh, R. R., Ashby, M., Tadich, P. V. (2019). Wearable computing apparatus and method.

Klappert, W. R., Nichols, M. R., Shimy, C., Wagner, W., Chen, Y., Stathacopoulos, P. T. (2013). Methods and systems for monitoring attentiveness of a user based on brain activity.

Sato, D., Sugio, T. (2010). Eye-gaze tracking device, eye-gaze tracking method, electro-oculography measuring device, wearable camera, head-mounted display, electronic eyeglasses, and ophthalmological diagnosis device.

1.-26. (canceled)

27. A method of detecting and tracking blink and blink patterns of a user, the method comprising:
placing an electronic device with a housing on a head of the user;
placing one or more biopotential sensors of the housing in contact with skin of the user;
detecting, using the biopotential sensors, signals related to eye blink of the user; and
inputting the signals into a model capable of decoding gaze and eyelid motion in real-time.

28. The method of claim 27, wherein the biopotential sensors are capable of detecting signals of electroencephalography (EEG), electro-oculography (EOG), and electromyography (EMG).

29. The method of claim 27, wherein the signals are collected from around the ears of the user are one or more of electroencephalography (EEG), electro-oculography (EOG), and electromyography (EMG) signals, and are processed to extract signals indicative of blink, and are in isolation or in combination with other signals including head direction, auditory attention, electrocardiography, temperature, blood oxygen level, activity level, and other bio-signals in order to further indicate blink.

30. The method of claim 27, wherein the housing containing the sensors is located on locations on the head of the user, including on the ear, in the ear, around the ear, horizontal to one or more of the left or right eye, vertical to one or more of the left or right eye, on the forehead, on one or more of the left and right temple, on the face, on the cheek, on the neck, or on the frontal or occipital regions of the head.

31. The method of claim 27, wherein the model is capable of detecting and discriminating between one or more different types of blink, including spontaneous blink, reflexive blink, voluntary blink, unilateral left blink, unilateral right blink, or bilateral blink.

32. The method of claim 27, wherein the model is capable of measuring characteristics of a blink, including blink duration, blink intensity, or blink velocity; and
wherein the model is capable of recognizing patterns and sequences of the signals.

33. The method of claim 27, wherein the inputted signals to the model include any one or combination of electroencephalography (EEG), electro-oculography (EOG), and electromyography (EMG) signals, head direction, auditory attention, electrocardiography, temperature, blood oxygen level, activity level, and other bio-signals derived from electrodes and sensors on the headworn device.

34. The method of claim 33, wherein the inputs comprise one or more of the following forms: raw data, filtered data, low-pass filtered data, bandpass filtered data, averaged data, subtraction of left from right data, subtraction of right from left data, subtraction of left average from right average, and subtraction of right average from left average.

35. The method of claim 33, one or more of the signals are a function (f) of one or more or an average of multiple signals from the left amplified voltages Vleft, signal=f(Vleft), or right amplified voltages Vright, signal=f(Vright) or from the difference between one or more or an average of multiple signals of the left and right amplified voltages Vleft and Vright, signal=f(Vleft—Vright).

36. The method of claim 27, wherein the model comprises signal processing methods or machine learning approaches that are based on linear or non-linear models such as artificial neural network models, and where the models can be deep and/or shallow digital and/or analog artificial neural network models.

37. The method of claim 27, wherein the model is a stand-alone blink model or a model in combination with other brain decoding models.

38. The method of claim 27, wherein the signals are used in a sensor fusion approach, in conjunction with other signals including electroencephalography (EEG), electro-oculography (EOG), and electromyography (EMG) signals, head direction, auditory attention, electrocardiography, temperature, blood oxygen level, activity level, and other bio-signals either in the same or in separate parallel algorithms, and from which the sensor fusion approach can determine additional insights or commands from the user.

39. The method of claim 27, further comprising assigning the blink or blink patterns to a user interface method including operating a click of a mouse, typing, toggling a switch, or selecting items.

40. The method of claim 27, further comprising assigning the blink or blink patterns to a predesignated command, including operating as a direction function to navigate menu items, selecting different desired sounds, confirming an action, dismissing a notification, changing audio settings, answering a phone, repeating a song, pausing audio, choosing between connected devices, or switching between pre-programmed settings.

41. The method of claim 27, further comprising providing control to connected electronic devices including mobile phones, smart watches, electric wheelchairs, audio devices, earphones, headphones, hearing aids, cochlear implants, computers, appliances, gaming devices, augmented reality devices, virtual reality devices, extended reality devices, machinery, vehicles, or electronics connected by wireless connectivity.

42. The method of claim 27, wherein the signals provide feedback from the user to a device, the feedback including approval of an action or disapproval of an action.

43. The method of claim 27, wherein characteristics of a blink are used to provide a further dimension of control.

44. The method of claim 40, wherein gaze direction indicates navigation in a virtual or physical environment, and blink detection is used as selection, operation, or execution.

45. The method of claim 38, wherein a blink with or without the one or more of the signals provide information regarding conditions of the user's physical health including fatigue, injury, illness or disease.

46. The method of claim 27, wherein blink data provides additional information about the state of an opening of the eye.
